(19) United States
(12) Patent Application Publication
(10) Pub. No.: US 2011/0236455 A1
(43) Pub. Date: Sep. 29, 2011

(54) Novel Regulators of the Innate Immune System

(76) Inventors: Peter Zipfel, Jena (DE); Christina Skerka, Jena (DE); Reinhard Wallich, Heidelberg (DE)

(21) Appl. No.: 13/121,029

(22) PCT Filed: Sep. 28, 2009
(86) PCT No.: PCT/EP09/06963

§ 371 Date: Mar. 25, 2011

(30) Foreign Application Priority Data
Sep. 26, 2008 (DE) 10 2008 049 136.5

(51) Int. Cl. A61K 38/17; C07K 14/46; A61M 31/00; C07K 16/18; C12N 5/16; G01N 33/577; A61P 29/00; A61L 33/12

(52) U.S. Cl. 424/423; 514/21.2; 530/350; 530/388.25; 435/346; 436/501; 427/2.24

(57) ABSTRACT

The present invention relates to the identification of novel regulators of the innate immune system, in particular the complement system. More particularly, the present invention relates to specific C5 convertase inhibitors. These novel inhibitors are particularly useful for treating inflammatory diseases involving the complement system. In a first aspect, the present invention focuses on the use of CFHR proteins and functional fragments or functional derivatives thereof for preventing inflammatory reactions. In a further aspect, the present invention focuses on the use of said CFHR proteins for inactivating complement activation during transplantation and dialysis and for coating devices which come into contact with blood or body fluids, in particular implants. The invention furthermore provides a pharmaceutical composition comprising functional CFHR protein in combination with functional factor H. In a further aspect, the present invention focuses on providing monoclonal antibodies which specifically detect CFHR proteins, and on the use thereof in methods of determining CFHR in body fluids. These methods are particularly suitable for diagnosing inflammatory diseases.

The present invention relates to the identification of novel regulators of the innate immune system, in particular the complement system. More particularly, the present invention relates to specific C5 convertase inhibitors. These novel inhibitors are particularly useful for treating inflammatory diseases involving the complement system. In a first aspect, the present invention focuses on the use of CFHR proteins and functional fragments or functional derivatives thereof for preventing inflammatory reactions. In a further aspect, the present invention focuses on the use of said CFHR proteins for inactivating complement activation during transplantation and dialysis and for coating devices which come into contact with blood or body fluids, in particular implants. The invention furthermore provides a pharmaceutical composition comprising functional CFHR protein in combination with functional factor H. In a further aspect, the present invention focuses on providing monoclonal antibodies which specifically detect CFHR proteins, and on the use thereof in methods of determining CFHR in body fluids. These methods are particularly suitable for diagnosing inflammatory diseases.

### PRIOR ART

The complement system is an important element in both innate and acquired immunity and is essential for causing a protective immune response to a foreign intruder. The alternative complement system pathway is activated spontaneously and comprises the formation of C3 convertase (C3bBb) which cleaves C3, the central component of the complement system. This cleavage generates the anaphylactic C3a peptide and the active protein or activation product, C3b, which can attach to a surface. C3b that has attached to foreign or modified surfaces binds factor B, thereby forming C3 convertase (C3bBb). The latter enhances further complement activation, ultimately leading to opsonization and phagocytosis of the intruding objects such as microbes. Binding of a second C3b molecule to said C3 convertase results in the formation of C5 convertase (C3bBbC3b) of the alternative pathway. C5 convertase cleaves C5 and generates the potent C5a chemoattractor and the C5b peptide. Said C5b peptide initiates formation of the terminal membrane attack complex (MAC). Owing to conformationary changes, C5b immediately binds to C6 and C7 in an enzyme-independent manner. This C5b67 complex formed detaches from the convertase and attaches to lipid bilayers. The complete terminal membrane attack complex is formed after binding of C8 and C9 and results in lysis of the pathogen and cells.

Once activated, this defense system is tightly regulated on the surface of host cells by both membrane-anchored and soluble regulators which attach both in the liquid phase and on the surface. This tight regulation is necessary in order to ensure that there are no adverse effects toward endogenous tissue and endogenous cells. Single mutations in genes coding for the corresponding host cell regulators and resulting in defective protein functions cause predisposition to various immune defects and autoimmune diseases and also renal and retinal disorders, for example hemolytic uremic syndrome (HUS), membranoproliferative glomerulonephritis type II (MPGN II) or age-related macular degeneration (AMD).

It is known that these different disorders are caused by defective local complement regulations and associated with genetic variations and mutations in complement components and regulators such as CFH (complement factor H). Thus a deletion of an 84 kb genomic fragment on human chromosome 1, which results in the loss of complement factor H-related genes 1 and 3 (CFHR1, CFHR3), was shown to be associated with both HUS and AMD (Zipfel PF. et al., PLoS .3:E41 (2007), Hughes A. E. et al.; Nat. Genet. 38, 1173-1177 (2006)). However, said chromosomal deletions exhibited opposite effects, thus, in the case of HUS, resulting in an increased risk of the disease, whereas a protective effect is described in the case of AMD.

The absence of these two plasma proteins, CFHR1 and CFH3, furthermore correlates with the presence of autoantibodies to CFH (Jozsi, M. et al., Blood, 111, 1512-1514 (2008)). These identified autoantibodies bind to the C terminals of CFH. This region is a focal point of HUS mutations, resulting in reduced attachment of CFH to the surface. Corresponding autoantibody binding to CFH therefore inhibits adhesion of CFH to the surface, thereby causing damage to endothelial cells as well as to platelets.

The family of CFHR proteins currently comprises five members in humans, CFHR1, CFHR2, CFHR3, CFHR4 and CFHR5. CFHR genes and proteins are also present in other species. In mice and rats, for example, they are referred to as CFHR-A, CFHR-B, CFHR-C, etc.

All of these members of the CFHR protein family are characterized by a very similar structure, structurally similar modules and a high sequence homology among the individual modules. However, each CFHR protein is encoded by a specific independent gene. The function of the individual CFHR proteins is not known. CFHR proteins have merely been shown not to have any cofactor activity and decay activity.

The CFHR proteins also have high sequence homology to complement factor H (CFH). CFHR1, for example, in particular in the C-terminal region with its three SCR (short complement regulator) domains, exhibits a high homology which varies from nearly 100% identity to a low, approx. 65% identity with the corresponding SCR domains at the C terminals of complement factor H.

The CFHR1 plasma protein is composed of 5 of these SCR domains and has been identified in two glycosylated forms in human plasma. CFHR1-beta has two, and CFHR1-alpha has one attached sugar side chain.

The function of CFHR-1 and the related molecules CFHR2, CFHR3, CFHR4 and CFHR5 is unknown. It has previously been speculated that said molecules might have the following functions: binding to C3b and to heparin, and a modulating influence on the regulatory function of factor H.

As discussed above, C5 convertase is an important target for inhibiting complement activation, since both the C5a anaphylatoxin produced from C5 and the resulting C5b peptide which, together with C6, forms the starting complex for forming the terminal membrane attack complex are required for triggering a local inflammatory response in the case of an infection.

Inhibitors which specifically inhibit this enzyme are not known to date and are therefore particularly suitable for inhibiting the corresponding subsequent alternative complement activation.

It is an object of the present invention to provide specific C5 convertase inhibitors in order to inhibit in this way the alternative pathway, i.e. complement activation including the formation of terminal membrane attack complexes, and to inhibit the formation of active anaphylactic, and possibly antimicrobial, peptides, namely C5a. Preferably, said specific inhibition of C5 convertase should not affect the classical pathway or the lectin pathway of complement activation.

In addition, there is great interest in inhibiting the terminal complement activation by way of forming and assembling the terminal complement complex (MAC, membrane attack complex, or TCC) and incorporating the latter into the lipid bilayer membrane. Two inhibitors of the terminal complement pathway are currently known, clusterin and vitronectin. However, specificity of these regulators is not very high, and it is therefore sensible to employ further more specific inhibitors.

Another object of the present invention is that of providing detection means, in particular antibodies, which allow CFHR molecules to be specifically detected. Finally, another object of the present invention is that of providing means for treating inflammations and also methods related thereto.

### SUMMARY OF THE PRESENT INVENTION

The present invention provides the use of CFHR proteins, more specifically of CFHR1 proteins, or of functional fragments or functional derivatives thereof for the treatment or prophylaxis of autoimmune diseases or inflammatory reactions.

According to the present invention, CFHR proteins were found to be specific inhibitors of C5 convertase. Specific inhibition of C5 convertase can inhibit both the formation of anaphylatoxin C5a and the formation of the terminal membrane attack complex. The CFHR proteins here are specific C5 convertase inhibitors which, in contrast to known C3/C5 convertase inhibitors such as CFH, do not inhibit C3 convertase. I.e., the present application describes, for the first time, a C5 convertase-specific inhibitor. These specific inhibitors allow the alternative pathway of complement activation to be modulated, without significantly affecting, for example blocking, the classical pathway.

In one aspect, the present invention focuses on the use of said functional CFHR proteins and of their functional fragments and derivatives for inactivating complement activation, in particular during transplantation or dialysis. In a further aspect, said functional CFHR proteins may be used for coating surfaces which may come into contact with blood and body fluid, such as implant surfaces.

A further aspect provides corresponding coatings and devices.

Furthermore, the present invention focuses on a pharmaceutical composition comprising functional CFHR protein in combination with functional factor H.

Finally, a monoclonal antibody is provided which specifically recognizes CFHR proteins. More specifically, said monoclonal antibody allows specific recognition of CFHR protein, for example CFHR1 protein over complement factor H.

Finally, the invention provides methods of determining CFHR in body fluids, more specifically in blood and blood plasma, comprising the use of the antibody of the invention, particularly for diagnosing hemolytic uremic syndrome, age-related macular degeneration or membranoproliferative glomerulonephritis, but also atheriosclerosis and other autoimmune diseases such as systemic lupus erythematosus.

### BRIEF DESCRIPTION OF THE FIGURES

FIG. 1: FIG. 1 depicts the ability of CFHR1 to bind to C3b, C3d, heparin and to human cells. FIG. 1a depicts the structure of CFHR1 in comparison with complement factor H. The first two SCRs of CFHR1 show 42 and 34%, respectively, sequence identity to SCRs 6 and 7 of CFH. The three C-terminal SCRs of CFHR1 show 100, 100 and 98%, respectively, sequence identity to the SCRs 18 to 20 of CFH. FIG. 1b indicates that equimolar concentrations of CFHR1 and CFH bind to immobilized C3b. The data show the average values plus minus standard deviations of one of three independent experiments. A490: Absorbance at 490 nm. Co: Control. FIG. 1c depicts the binding of CFHR1 and CFH to immobilized heparin. The control represents background binding of the antibodies in the presence of the buffer. FIG. 1d depicts CFHR1 binding to HUVEC cells, with the CFHR1 protein having been obtained from plasma. The cells were incubated in human plasma, and bound CFHR1 was detected with the aid of the monoclonal antibody JHD10, using flow cytometry. The control cells were treated with secondary antibodies alone. FIG. 1e depicts the binding (10 μg/ml) of recombinant CFHR1 to HUVEC cells and to retinal pigment epithelial cells; the line corresponds to a size of 20 μm. Similarly, CFHR1 binding was studied with rabbit erythrocytes pretreated with C3b.

FIG. 2 depicts CFHR1 staining on tissue sections of the kidney and the choroid. I depicts staining in the kidney, II depicts staining in the eye. Counterstain is with propidium iodide.

FIG. 3 depicts the specificity of the monoclonal antibody described herein, JHD10, which specifically recognizes CFHR1 in human serum and does not cross react with CFH. FIG. 3a depicts the specificity of the monoclonal antibody. Lane 1: normal human serum, lane 2: CFH, lane 3: normal human serum, lane 4: CFH. While the CFH-specific antibody detects CFH, the monoclonal JHD10 antibody shows reactivity only toward the CFHR1 molecules. FIG. 3b depicts a silver stain of purified recombinant CFHR1 (lane 1) and CFHR fragments CFHR1/1-2 (lane 2) and CFHR1/3-5 (lane 3). FIG. 3c depicts recombinant CFHR1 and plasma-derived native CFHR1 (lanes 1 and 3 and, respectively, 2 and 4). The right-hand side shows an immunoblot using the specific CFHR1 antibody, with the left-hand side showing a silver stain.

FIG. 4 illustrates that CFHR1 competes with factor H for binding to the binding partners. FIG. 4a shows immunofluorescence images which reveal that CFH and CFHR1 colocalize (yellow signal resulting from green CFH fluorescence and red CFHR1 fluorescence). FIG. 4b depicts the competition of CFHR1 with factor H for C3 binding. The data are average values from three separate experiments plus minus standard deviations. A490: Absorbance at 490 nm; *p<0.05 in respect of binding of CFHR1: CFH (0:1). FIG. 4c depicts competitive binding of CFHR1 with factor H with heparin. The mobility of the alpha′ and beta chains and of the degradation fragments are shown.

FIG. 5 depicts the results with regard to regulation of the alternative complement pathway by CFHR1. FIG. 5a illustrates hemolysis of sheep erythrocytes in the presence of CFHR1 and CFH-depleted normal human serum (HPΔCFH-CFHR1). The results with vitronectin, CFH and HSA are shown for comparison. The addition of CFHR1 was shown to cause a dose-dependent reduction in lysis. FIG. 5b illustrates inhibition of the alternative complement pathway by CFHR1. Each of the three complement pathways was induced separately with the aid of an ELISA. AP: alternative pathway, CP: classical pathway, LP: lectin pathway; A440: absorbance at 440 nm. With the alternative pathway, a dose-dependent action was shown for CFHR1 (squares). FIG. 5c indicates that CFHR1, in contrast to CFH, does not affect C3a formation. FIG. 5d illustrates the protective action of CFHR1 and inhibition of C5bC6 generation. For this purpose, rabbit erythrocytes were incubated with human complement-active, C7-depleted CFHR1/CFHR3-deficient human plasma. CFHR1 was then added at the corresponding concentration. Lysis was carried out with the aid of chicken erythrocytes to which sublytical amounts of human plasma as source of C7 were added. Erythrocyte lysis was determined after 15 minutes of incubation.

FIG. 6 illustrates that CFHR1 regulates C5 convertase activity and can bind to C5 and C5b6, and also inhibits binding of C5b6 to cell surfaces and thus MAC formation and enzymic MAC activity. FIG. 6a illustrates CFHR1 action on the lysis of sheep erythrocytes and formation of C3a and C5a in the supernatant of the sample. The data are average values of three separate experiments and their standard deviations. *:p<0.05, **:p<0.005, compared to the control. FIG. 6b determines the effect of CFHR1 on the deposition of C3b and C5b on the surface of sheep erythrocytes. FIG. 6c shows immuno-fluorescence images which reveal that CFHR1 inhibits C5b attachment on cell surfaces, while CFH inhibits the attachment of both C3b and C5b.

FIG. 7 indicates binding of CFHR1 to C5 and C5b6, respectively, and inhibition of the terminal complement pathway. FIG. 7a illustrates binding of CFHR1 to immobilized C5 and C5b6, respectively. Binding of the JHD10 antibody to C5 or C5b6 alone was determined by way of a control. FIG. 7b illustrates that binding of C5 and C5b6, respectively, occurs via the N-terminal region of CFHR1. **p<0.001 with respect to binding of C5 or C5b6 to C18-mediated CFHR1 immobilization. In FIG. 7c, chicken erythrocytes were incubated with C5b6 complexes (5 ng/ml), with increasing concentrations of CFHR1 and non-lytic HP being added. A dose-dependent inhibition of lysis can be seen with the addition of CFHR1, while the controls CFH and HSA do not restrict lysis. FIG. 7d illustrates inhibition of lysis by CFHR1 of sheep erythrocytes in comparison with CFH and BSA. Similar values were found for the known inhibitor vitronectin (Vitro). The data are average values plus minus standard deviations of 2 separate experiments.

### DETAILED DESCRIPTION OF THE INVENTION

In a first aspect, the present invention relates to the use of functional CFHR proteins for the treatment or prophylaxis of autoimmune diseases and inflammatory reactions.

In this context, the expression “functional CFHR proteins” means both the CFHR protein itself and functional fragments of said protein and also functional derivatives of the CFHR protein which are similar to complete CFHR protein in respect of their function of inhibiting C5 convertase, as demonstrated herein. The expression CFHR protein here includes the human CFHR proteins CFHR1, CFHR2, CFHR3, CFHR4 and CFHR5, with the individual proteins having the following accession numbers: CFHR1 NM 002113 gi 118442838, CFHR2 NM 005666 gi49574530, CFHR3 NM 021023 gi 118421081, CFHR4 NM 006684 gi 117320517, CFHR5 NM 030787 gi 164607154.

The expression “functional CFHR protein” means hereinbelow both the protein and the functional derivatives and functional fragments, insofar as is stated otherwise. Preference is given, however, to using the mature CFHR protein.

“Functional” here means that the CFHR molecules, more specifically the CFHR proteins but also the fragments or derivatives of said CFHR proteins, act in an inhibiting manner on C5 convertase and, where appropriate, inhibit MAC formation and activity without inhibiting the C3 convertase of the complement system, as demonstrated here for CFHR-1. Preference is given here to the functional fragment having at least 60%, such as 70%, in particular 80%, preferably 90%, activity with regard to the inhibitory action on C5 convertase compared to the complete CFHR protein, more specifically the CFHR1 protein.

Fragments here include those polypeptides derived from the CFHR protein which have at least one amino acid deletion, mutation or addition.

Derivatives of the CFHR protein are polypeptides which have been modified by posttranslational modification of the CFHR protein or CFHR fragment, such as glycosylation, acetylation, phosphorylation and the like, for example. Said derivatives furthermore include those polypeptides in which one or more amino acid analogues, including amino acids that do not occur naturally, polypeptides with substituted nomenclatures, such as PNA polypeptides, and further modification, as occur naturally or non-naturally.

Unless stated otherwise, the expression “complement activation” means firstly the formation of the terminal membrane attack complex, also referred to as MAC hereinbelow. Secondly, the expression “complement activation” comprises the generation of inflammation-mediating peptides, in particular anaphylatoxins such as C5a.

The expression “body fluids” means bodily fluids including blood, blood plasma, lymph, cerebrospinal fluid, CSF, synovial fluid, etc.

Preference is given according to the invention to the CFHR protein being the CFHR1 protein.

Functional CFHR proteins were shown to exhibit an inhibitory action specifically on C5 convertase. C5 convertase cleaves the C5 complement to give the peptide components C5a and C5b. C5a acts by way of an inflammation-mediating anaphylatoxin, in locally inducing or amplifying the inflammatory reactions. A C5b molecule binds to a C6 molecule, and this C5b,6 complex then attaches to one molecule of C7. This reaction results in a conformational change in the molecules involved, with a hydrophobic site on C7 becoming accessible. This hydrophobic C7 domain moves into the lipid bilayer. Hydrophobic sites are exposed similarly in the later components, C8 and C9, when they bind to the complex, allowing them to likewise enter the lipid bilayer. The next step comprises a C8 molecule attaching to the membrane-associated C5b67 complex. C8 is a complex of two proteins: C8beta which binds to C5b, and C8alpha-gamma which enters the lipid bilayer. Finally, C8alpha-gamma induces polymerization of from 10 to 16 C9 molecules to give an annular structure which is referred to as terminal membrane attack complex. In this way it is possible to finally destroy the cell or pathogen.

The present application now indicates that CFHR protein, more particularly the CFHR1 protein, can inhibit the activity of C5 convertase and, furthermore, formation of C5a and C5b. The CFHD protein was furthermore shown according to the invention to not inhibit the activity of C3 convertase. Finally, the CFHR protein enables the formation of the C5Bb6(7) complex and thus MAC formation to be inhibited. This property allows the functional CFHR protein to be used for treating or preventing inflammatory reactions. Said inflammatory reactions are preferably inflammatory reactions in the course of an autoimmune disease.

These autoimmune diseases in which autoantibodies may be involved are more specifically such diseases as rheumatoid arthritis, lupus erythematosus, hemolytic uremic syndrome, atheriosclerosis, renal disorders such as glomerulonephritis, and others.

Furthermore, conditions associated with proteinuria, such as proteinuria with renal disorders, can be treated.

The inflammatory reactions to be treated are particularly preferably those in the course of sepsis, rheumatoid arthritis, Alzheimer's disease, atheriosclerosis, lupus erythematosus, antiphospholipid syndrome, preeclampsia, multiple sclerosis, myocarditis, asthma, recurrent pregnancy loss syndrome.

In a further aspect, the CFHR proteins are suitable for inactivating complement activation and, more specifically herein, for inactivating complement activation during transplantation or dialysis. Since CFHR1 deficiency results in kidney damage and the activated complement system plays an important part in transplant acceptance during transplantation, specific inhibitors which enable complement activation to be specifically inhibited are therapeutically desirable.

Finally, a further use of the CFHR protein relates to the coating of devices and surfaces that come into contact with body fluids and in particular blood or blood plasma. Coating of said devices may prevent complement activation in the body fluid by these devices.

A further aspect of the present invention therefore focuses on a coating of a device which comes into contact with body fluids, in particular blood or blood plasma, characterized in that the surface is coated with functional CFHR proteins.

Said coating is particularly suitable for implantable devices, said implantable device being intended for use on the body of an individual.

A further aspect of the present invention focuses on those devices which come into contact with body fluids such as in particular blood or blood plasma. Said device which is in particular implantable devices stands out due to the fact that functional CFHR protein has been applied to its surface.

CFHR1, as an example of CFHR proteins, exhibits an inhibitory action on complement activation of the alternative pathway. In this context, said action was found to not be based on inhibition of C3 convertase, although CFHR1 competes with complement factor H (CFH) for binding to C3b and can partly replace CFH. To the contrary, inhibition of C3 convertase is not observed. Experiments showed that CFHR1 can regulate activation of the alternative complement pathway in an essentially specific manner.

A CFHR1 dose-dependent inhibiting effect on complement-mediated lysis due to formation of the terminal complex and MAC formation was demonstrated. That is to say, the CFHR molecule according to the invention can inhibit the formation and activity of MAC.

Finally, CFHR1 was shown to be able to inhibit, via C5 convertase and the MAC, especially the alternative pathway, but possibly also the classical and lectin pathways.

The addition of CFHR1 resulted in down-regulated production of C5a peptides and C5b proteins; however, generation of C3a and C3b was not affected. In contrast, the action of CFH which inhibits both C3 convertase and C5 convertase and therefore inhibits generation both of C3a and C3b and of C5a and C5b. That is to say, the CFHR proteins show an activity profile which differs from CFH by acting in an inhibitory manner only on C5 convertase, while CFH has an effect on C3 convertase which is upstream in the cascade and on C5 convertase.

CFHR1 is actually capable of binding to C5 and to the C5b6 complex, thereby inhibiting formation of the MAC and preventing lysis of cells and/or microbes.

CFHR1 was thus shown to regulate the alternative pathway by inhibiting C5 convertase activity, assembling of the MAC and by preventing surface binding.

Another aspect of the present invention focuses on supplementing a CFH therapy with CFHR proteins. The two proteins are important regulators of the complement cascade which lead to different interventions into the complement system.

A further aspect of the present invention therefore focuses on a pharmaceutical composition comprising functional CFHR protein. Said pharmaceutical composition preferably comprises furthermore the functional complement factor H, i.e. the pharmaceutical composition, in a preferred embodiment, is one that comprises functional CFHR protein in combination with functional complement factor H. The pharmaceutical composition furthermore optionally includes customary pharmaceutically acceptable diluents, carriers and excipients which as such are well known to the skilled worker.

Contrary to previous thinking, CFH and CFHR protein have different actions, and it is accordingly sensible to combine these two classes of proteins in a pharmaceutical composition in order to utilize the different effects described herein of said molecules. Thus, contrary to the teaching in WO2006/088950, CFH cannot be replaced with CFHR, due to supposedly having the same action, but rather a combination of these two molecular classes results in an improved action, since they exhibit different actions on the complement system.

The pharmaceutical formulations may furthermore comprise customary pharmaceutically acceptable excipients such as are well known to the skilled worker. Said pharmaceutical formulations may be administered via the usual routes and comprise effective amounts of the active ingredients, namely functional CFHR protein, where appropriate in combination with functional complement factor H.

Functional CFHR proteins and functional complement factor H may be obtained from natural sources such as human plasma or serum or recombinantly. The skilled worker is familiar with methods of isolating natural CFHR protein and/or factor H protein and with genetic engineering methods of recombinantly producing said functional CFHR proteins and functional factor H proteins.

Acceptable pharmaceutical carriers and excipients as well as suitable pharmaceutical formulations are generally known, see, for example, pharmaceutical formulation development of peptides or proteins, Frokjaer et al. Taylor & Francis (2000) or Handbook of Pharmaceutical Excipients, 3^{rd} Edition, Kibbe et al., Pharmaceutical Press (2000).

For example, the pharmaceutical formulations may be provided by way of a lysed or stabilized soluble form.

The routes of administration comprise systemic routes of administration such as parenteral routes of administration, for example intravenous, subcutaneous, intramuscular, intraperitoneal, intracerebral, intra-pulmonary, intranasal or transdermal routes or enteral routes such as oral, vaginal or rectal routes.

The therapeutically effective amount of functional CFHR protein or functional factor H protein may depend on many factors including indication, formulation, route of administration and age and state of the individual. The skilled worker may determine the effective dose by taking into account said parameters.

The pharmaceutical compositions according to the present invention may be administered alone or in conjunction with other therapeutic means which may optionally be incorporated into a pharmaceutical formulation.

Finally, in a further aspect, the present invention focuses on monoclonal antibodies which specifically detect CFHR protein immunohistologically and immunobiochemically. More specifically, these monoclonal antibodies allow CFHR protein and CFH protein to be distinguished.

Said monoclonal antibody is more specifically one which exhibits specifically binding to human CFHR, in particular CFHR1 protein, and which may have the same properties as the monoclonal antibody of the JHD-7.10.1 hybridoma cell line, deposited in accordance with the Budapest Treaty with the DSMZ, Brunswick, Germany. This monoclonal antibody binds in the N-terminal region of the CFHR-1 protein.

In a preferred embodiment, the present invention therefore focuses on a monoclonal antibody capable of specifically binding to human CFHR protein, wherein said monoclonal antibody reacts with the same epitope of human CFHR protein as the monoclonal antibody that can be obtained from the JHD-7.10.1 hybridoma cell line, deposited in accordance with the Budapest Treaty with the DSMZ, Brunswick, Germany, DSM ACC 2978.

Another embodiment of the present invention relates to a hybridoma cell line expressing a monoclonal antibody of the invention. Particular preference is given to said hybridoma cell line being the JHD-7.10.1 hybridoma cell line, deposited in accordance with the Budapest Treaty with the DSMZ, Brunswick, Germany, DSM ACC 2978.

These monoclonal antibodies make possible methods of specifically determining CFHR protein, in particular CFHR1 protein, in body fluids such as blood, in particular plasma or serum, without determining CFH proteins. This method of the invention comprises the step of incubating a sample to be tested with a monoclonal antibody of the invention and determining the monoclonal antibodies bound to CFHR, in particular CFHR1 protein. Said method may be a quanlitative or (semi-)quantitative method.

Such diagnostic methods are particularly suitable for determining the CFHR1 protein content in body fluids such as the blood and in particular the plasma of individuals who are examined for hemolytic uremic syndrome, age-related macular degeneration or for membranoproliferative glomerulonephritis and other inflammation-based renal disorders and also other autoimmune diseases. CFHR1 deficiency, but also CFHR3 deficiency, in the plasma is a risk factor in respect of developing HUS, for example. An antibody of the invention for detecting CFHR1 in plasma is therefore suitable for determining this risk and therapeutically useful when said deficiency has been established and especially with young patients suffering from HUS, since said deficiency correlates with the occurrence of autoantibodies, which initiates a different type of treatment of this patient, namely reduction of said antibodies and complementation of the CFHR proteins.

In a preferred embodiment, the present invention therefore relates to a method of diagnosing HUS, comprising the steps of determining CFHR protein with the aid of the antibody of the invention in combination with determining autoantibodies.

The diagnostic methods of the invention may include customary methods such as, for example, ELISA, Western blot methods, rapid immunological tests, and protein-based microarrays.

The presence of CFHR1 in plasma is also important in age-related macular degeneration (AMD), the development of AMD correlating with the presence of CFHR1 protein. A corresponding diagnostic method therefore facilitates AMD recognition and prognosis.

Further aspects of the invention focus on methods of treating inflammatory reactions with functional CFHR proteins but also with nucleic acids coding therefor.

The invention will be further illustrated hereinbelow on the basis of examples using CFHR1 protein. The invention is not limited to the examples below, however.

### Methods

### Proteins and Antibodies

Recombinant CFHR1 and deletion mutants of CFHR1 SCRS1-2 (CFHR1/1-2) and CFHR1SCR3-5 (CFHR1/3-5) were expressed according to known methods. The proteins were expressed in Pichia pastoris and purified with the aid of nickel chelate affinity chromatography. Vitronectin was obtained from BD Biosciences (Belgium). C3b, C3d, C5, C5bC6, factor H and factor I were obtained from Merck Biosciences (Schwalbach, Germany), and C7, C8 and C9 were obtained from Comptech (Taylor, USA).

### Purification of Natural CFHR1

CFHR1 protein was purified with the aid of heparin chromatography (HiTrap Heparin HP column, GE Healthcare, Munich, Germany), diluted with Sterofundin (Braun Melsungen, Germany). The proteins were eluted in a 3 step gradient of NaCl (100, 200 and 300 mM) dissolved in Sterofundin. The pooled eluate fractions of 100 and 200 mM NaCl gradients were concentrated (Superdex 2000, Satorius, Germany), and the proteins were adjusted with 1×PBS to pH 4.7. The samples were fractionated by means of SDS PAGE, and the band containing CFHR1 was identified based on the mobility of the size markers, the corresponding region was excised and CFHR1 was eluted, concentrated and dialyzed 6× against PBS, pH 4.7.

The mouse monoclonal antibody C18 (Alexis, Lausen, Switzerland) was employed for detecting the C terminus of CFH and CFHR1. The monoclonal JHD10 antibody from the JHD-7.10.1 hybridoma cell line, deposited in accordance with the Budapest Treaty with the DSMZ, Brunswick, Germany, DSM ACC 2978, was employed in order to specifically detect CFHR1. Said antibody is directed to purified CFHR1-SCR1-2 fragment, i.e. binds in the N-terminal region of CFHR1. At higher concentrations of this antibody, CFHR5 and CFHR2 are likewise detected. Anti-C5, anti-C6, anti-C7 were obtained from Comptech (USA).

Secondary antibodies were obtained from Dako (Glostrup, Denmark).

### Serum Samples

Normal human serum was obtained from healthy volunteers. Blood was taken from said healthy volunteers and stored at −80° C. until used. Six patients suffering from atypical HUS were likewise tested. CFHR1 deficiency was determined with the aid of Western blot analysis and verified by genetic analysis, as is known. CFH autoantibodies were identified by means of ELISA against CFH-specific antibodies, as described. For CFH depletion, 150 μl of protein A Sepharose were used as matrix (GE-HealthCare, Freiburg, Germany). The latter was incubated with 300 μg/ml monoclonal antibody C18 (Alexis, USA) and 150 μg/ml monoclonal antibody B22, from our own group, at 4° C. overnight. Depletion of the serum was verified with the aid of Western blot analysis. For C7 depletion, 150 μl of polyclonal goat anti-C7 (Quidel, USA) were used, and depletion was carried out as described for CFH.

The following sera were used: human complement active plasma (HP), complement active HP, from which CFH and CFHR1 were removed (HP_{ΔCFH}), complement active HP, with CFH, CFHR1 and C7 being removed (HP_{ΔCFHΔC7}), complement active plasma of CFHR1/CFHR3 deficient healthy persons (defHP), complement active defHP in which CFH and C7 were removed (defHP_{ΔCFHΔC7}).

### Cell Culture and Confocal Microscopy

Endothelial cells from human umbilical cord (HUVEC, ATCCF, CRL-1730) and retinal pigment epithelial cells (ARPE-19, ATCC CRL-2302) were cultured according to known methods. For binding experiments, the cells were incubated in serum-free medium for 24 hours, detached by means of brief incubation in 0.02% Accutase (PAA, Parching, Germany) at 37° C. and resuspended in PBS to which 1% BSA had been added. Sheep, rabbit and chicken erythrocytes were obtained from Rockland (USA). For confocal microscopy, HUVEC and ARPE-18 cells were grown on coverslips (Nunc) and washed with PBS, and unspecific binding sites were blocked with PBS to which 1% BSA had been added. The cells were then incubated with CFHR1 or CFH (100 μg/ml) or normal human plasma (NHP, 5%) for 60 minutes. CFHR1 binding was visualized with the aid of the monoclonal JHD10 antibody, with a secondary antimouse antibody labeled with Alexa 647, and CFH binding was visualized with a polyclonal antiserum specific for the N-terminal domain of CFH (anti-SCR1-4) (Alexis, USA), together with a secondary goat anti-rabbit antibody labeled with Alexa 488. The samples were washed with 1% BSA/PBS, counterstained with DAPI and wheatgerm agglutinine Texas Red 595, and studied with the aid of an LSM 510 META laser scanning microscope (Zeiss, Jena, Germany). The rabbit erythrocytes were incubated with 3b (10 μg/ml) prior to addition of CFHR1 (100 μg/ml), immobilized on a coverslip and stained with the monoclonal JHD10 antibody. Unspecific binding of the antibodies to the cells was eliminated, and no signals were detected in the absence of CFHR1 or CFH.

### Immunohistochemistry

Immunohistochemistry was carried out using two human donor eyes for which there were no clinical documents on early AMD and no documents on morphological evidence of ocular disorders. The donor eyes were obtained during autopsy and were processed within 15 hours post mortem. Furthermore, normal kidney tissue was obtained from two human adult donor kidneys which had not been used for transplantation. Posterior eye specimens and part of the decapsulated kidney were embedded in OCT compound and frozen in isopentane-cooled, liquid nitrogen. Cryostat sections (6 μm) were fixed in cold acetone, blocked with 10% normal goat serum and incubated with a mouse monoclonal antibody to CFHR1 (JHD10), diluted 1:100 in PBS, at 4° C. overnight. Antibody binding was detected with the aid of an Alexa 488-conjugated secondary antibody (Molecular Probes, Eugene, USA). Nuclear counterstaining was carried out using propidium iodide. For preabsorption experiments, the primary antibody was treated either with CFHR1 or with CFH for one hour.

### Binding of CFHR1 to Heparin, C3b, C5 and C5bC6

MaxiSorp plastic dishes (Nunc) were coated with heparin (heparin sodium salt; Sigma, Germany), 500 units/plate, or C3b, C3d (Merck Biosciences, Germany) (10 μg/ml), C5 or C5bC6 (Merck Biosciences, Germany), (5 μg/ml), and unspecific binding sites were blocked with 2% BSA in PBS. CFHR1 (at various concentrations from 10 to 90 μg/ml) or CFH (75 μg/ml) were dissolved in binding buffer B (10 mM Na₂HP₄, 27 mM KCl, 1.4 M NaCl, 2% BSA, pH 7.4), added to each plate, and bound CFHR1 was detected using the monoclonal antibody C18, and bound CFH was detected using the antiserum of SCRs 1-4 in combination with the corresponding secondary HRP-conjugated rabbit anti-goat or anti-mouse IgG (Dako, Denmark, 1:4000 dilution). By way of a control, buffer B was added directly in the absence of the primary protein. JHD10 or mAk C18 (15 μg/ml) were incubated in parallel in a microtiter plate and used for capturing CFHR1 (30 μg/ml). After washing, C5 or C5bC6 (5 μg/ml) in gelatin veronal buffer (Sigma, Germany) was added, and bound proteins were identified using a monoclonal C5 antibody (Merck Biosciences, Germany). CFHR1-specific antiserum was employed in order to confirm binding of CFHR1 to the immobilized monoclonal antibodies. For competition experiments, C3b (10 μg/ml) was immobilized in a microtiter plate (Nunc, Germany), and constant amounts of CFH (5 μg/ml) were added. Furthermore, increasing amounts of CFHR1 (1.3 to 26.6 μg/ml), dissolved in buffer B, were added and bound CFHR1 and CFH were detected with monoclonal JHD10 antibodies or polyclonal antiserum to SCRs 1-4 of CFH.

### Cofactor Assays

Cofactor activity of heparin-bound CFH was measured by measuring the factor I-mediated degradation of C3b with the aid of Western blot analysis. Briefly, heparin (Fluka, Buchs, Switzerland) (5 μg/ml) was immobilized in a microtiter plate (EprarEx™, Plasso) at room temperature overnight, and any unspecific binding was blocked with 1% BSA in blocking buffer (20 mmol HEPES, 130 mmol NaCl, 0.05% Tween) at room temperature for two hours. For competition analyses, immobilized CFH was incubated with increasing amounts of CFHR1 (0.13 μg to 13.3 μg), followed by incubation with 2 μg of C3b and 0.28 μg of CFI in a total volume of 50 μl at 37° C. for 15 minutes. The samples were removed from the microtiter plates and incubated with sample buffer containing beta-2-mercaptoethanol and boiled at 95° C. for 5 minutes. The proteins were fractionated on a 10% SDS page, blotted onto a nitrocellulose membrane and developed with the aid of goat anti-human C3 (Calbiochem, 1:1000) and HRP-conjugated rabbit anti-goat Ig (Dako, 1:1000) according to general methods. The presence of the α′43 degradation band of C3b was determined with the aid of densitometry.

### ELISA

In order to determine whether there are CFHR1 effects on complement activation, CFHR1 activity on each of the three complement pathways was studied with the aid of WiELISA (Wieslab, Lund, Sweden). CFH and CFHR1-depleted NHS (1% classical and lectin and 20% alternative pathway) were incubated with increasing concentrations of CFHR1 (20 to 80 μg/ml) on ice for 10 minutes. Equal amounts of DPBS were added to each sample in order to eliminate buffer effects. Following incubation, the samples were treated according to the manufacturer's instructions.

In order to study CFHR1 regulation of C3 convertase, C3 convertase was generated by incubating C3b (2 μg/ml) and C3 (80 μg/ml) with factor D (4 μg/ml) and factor B (40 μg/ml) in activation buffer (20 mM Hepes, 144 mM NaCl, 7 mM MgCl₂, 10 mM EGTA, pH 7.4). C3 convertase activity was determined by way of C3a formation after incubating constant amounts of C3 (18 μg/ml) and increasing amounts of CFHR1 (25 and 50 μg/ml) or CFH (50 μg/ml) or 25 μg/ml human serum albumin (HSA). C3a concentrations were determined with the aid of ELISA (Quidel, USA) according to the manufacturer's instructions.

### Erythrocyte Lysis Assay

Sheep erythrocytes were incubated with 30% v/v CFH and CFHR1-depleted human plasma in AP buffer (20 mM Hepes, 144 mM NaCl, 7 mM MgCl₂, 10 mM EGTA, pH 7.4). The depleted plasma was tested for hemolysis of the sheep erythrocytes prior to the experiment. Hemolytic experiments were assayed in HEPES/EGTA buffer (20 mM Hepes, 144 mM NaCl, 7 mM MgCl₂, 10 mM EGTA, pH 7.4). Increasing concentrations of CFHR1 (5 to 160 μg/ml) were added to the plasma and incubated with approx. 2×10⁷ sheep erythrocytes at 37° C. for 15 minutes. Following incubation, the mixture was clarified by centrifugation and absorbance was measured at 415 nm in the supernatant. Furthermore, depleted plasma samples were incubated with equal amounts of CFH, Vitronectin or BSA. In one experiment, formation of complement activation products C3a and C5a was determined with the aid of ELISA. An aliquot of each sample was removed from the hemolysis assay and immediately diluted in ice-cold Hepes/EGTA buffer (C3a: 1:4000, C5a: 1:100) and stored on ice. C3a and C5a concentrations were measured by commercially available ELISAs according to the manufacturer's instructions (C3a: Quidel, USA, C5a: DRG Diagnostics, Marburg, Germany).

CFHR1- and CFHR3-deficient HP was depleted from the C7 complement component in order to prevent formation of the terminal membrane attack complex (MAC). Polyclonal C7 antiserum (Comtech, USA) was coupled in 1 ml of protein A Sepharose column and incubated with CFHR1- and CFHR3-deficient plasma. To this depletion serum, 25 μg/ml or 50 μg/ml recombinant CFHR1 were added and incubated with 2×10⁷ rabbit erythrocytes in Hepes/EGTA buffer over a period of from 5 to 30 minutes in order to activate the alternative complement pathway. Aliquots were taken from this activated plasma every 2.5 minutes, complement activation was stopped with the aid of a protease inhibitor (Complete, Roche, Germany), and hemolytic activity was determined by incubation with a small amount of deficient HP (1%) as source for C7-C9 and 2×10⁷ chicken erythrocytes. Hemolysis was determined for each activation time point by measuring absorbance at 415 nm.

Hemolysis of the chicken erythrocytes was determined in CFHR1/CFHR3-deficient, complement-inactivated (20 mM Hepes, 144 mM NaCl, 10 mM EDTA, pH 7.4) HP, with constant concentrations of C5b6 (5 ng/ml) protein complexes and increasing amounts of CFHR1 (25 to 100 μg/ml). The deficient plasma was incubated with C5b6 and CFHR1 at 37° C. for 15 minutes, and lysis of the chicken erythrocytes was determined by examining the supernatant at 415 nm. In order to demonstrate specificity of the CFHR1 function, deficient serum was incubated in parallel with equal amounts of CFH or BSA in the presence of C5b6. The activity of plasma-derived CFHR1 was tested in the same hemolytic assay. C5b6 complexes (5 ng/ml) were preincubated either with plasma-derived CFHR1 (0.75 μg/ml) or with recombinant CFHR1 (5 μg/ml) in 20 mM Hepes, 144 mM NaCl, 10 mM EDTA, pH 7.4 at 20° C. for 5 minutes. Sheep erythrocytes (2×10⁷) were added and, after 10 minutes at 20° C., the terminal components C7 (final concentration 1 μg/ml), C8 (0.2 μg/ml) and C9 (1 μg/ml) were added. The release of hemoglobin was measured at 415 nm after incubation at 37° C. for 30 minutes. The effect of the inhibitor of the terminal pathway, Vitronectin (1.25 μg/ml), and of BSA (1.25 μg/ml) was tested in the same way.

### Flow Cytometry

Flow cytometry was used in order to study the effect of CFHR1 on C5 deposition on erythrocytes during complement activation. To enable complement activation but prevent hemolysis, CFHR1/CFHR3-deficient plasma was treated by immunoaffinity chromatography to remove CFH and C7 (defHP_{ΔCFHΔC7}). Sheep and rabbit erythrocytes were incubated for each time point in 30% v/v defHP_{ΔCFHΔC7} in the presence or absence of 50 μg/ml CFHR1. To prevent too rapid degradation of the C3/C5 convertase complex, manitol replaced was substituted for sodium chloride in the buffer (20 mM Hepes, 250 mM manitol, 8 mM MgCl₂, 10 mM EGTA, pH 7.4). At each time point, the sample was transferred into ice-cold modified buffer containing 1% w/v BSA. To inhibit complement activation, the buffer contained a protease inhibitor mixture (Complete Inhibitor Mix, Roche, Germany). C5 was detected using a monoclonal mouse anti-C5 antibody (Quidel, USA). The erythrocytes were measured by means of a B&D LSR II using suitable laser and filter settings. 50 000 events were routinely counted. Binding of the serum-derived CFHR1 to HUVEC cells was tested by incubating said HUVEC cells, which were kept serum-free for 3 days, in 25% normal human serum for 30 minutes. The cells were washed, and CFHR1 binding was determined using the monoclonal JHD10 antibody.

Coating of Surfaces with CFHR1

CFHR1 may be bound either as recombinant protein or after purification from plasma on surfaces by standard methods such as, for example, in ELISA mixtures. For this purpose, CFHR1 is incubated with said material in a buffer solution and then washed thoroughly with a physiological buffer. An alternative possibility is directed immobilization by monoclonal antibodies such as, for example, the CFHR1-specific mAB JHD 10 or else C18, or polyclonal antisera. Another way of attachment is that of coating the surface with CFHR1 ligands such as heparin, for example. Since CFHR1 binds to heparin and other ligands, coating of the surface with CFHR1 is feasible in this way.

### Statistical Analysis

Statistics were carried out with the aid of Students' T test. P values of less than 0.05 were considered significant.

### Results

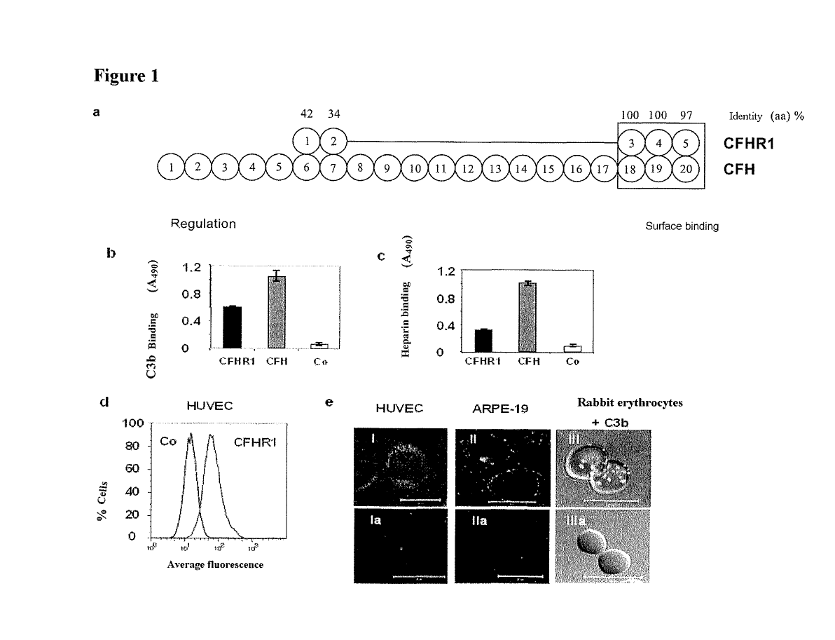

The experiments demonstrated that CFHR1 binds to C3b, C3d, heparin to human cells, as depicted in FIG. 1.

CFHR1 utilizes the C terminus both for C3b binding and heparin binding, since only the SCRs3-5 deletion mutants but not the SCRs1-2 deletion mutants bind to immobilized C3b and heparin. CFHR1 was furthermore found to bind to cell surfaces, see FIGS. 1d to e.

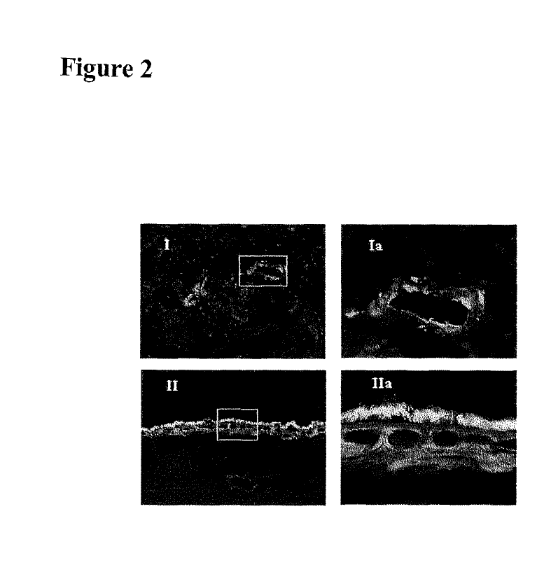

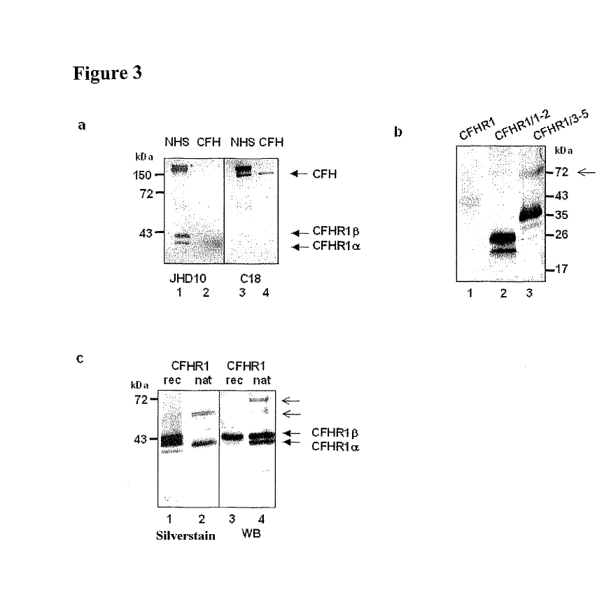

FIG. 2 depicts CFHR1 staining on tissue sections of kidney and retinal tissue, using here the novel monoclonal JHD10 antibody disclosed herein which recognizes an N-terminal epitope of CFHR1 protein, see FIG. 2. The specificity of said antibody is illustrated in FIG. 3.

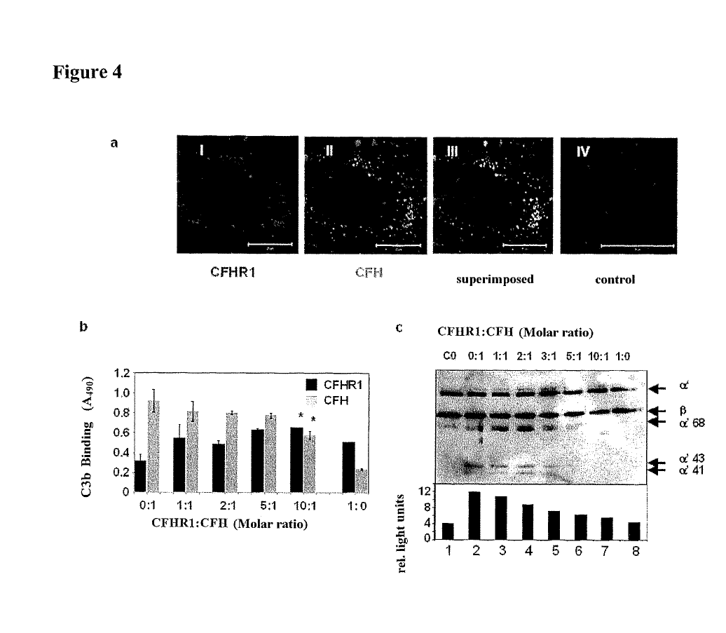

CFHR1 competes with CFH for the same binding sites. As illustrated in FIG. 4, the two molecules compete for binding to C3b, with CFHR1 being able to replace CFH and reduce CFH-mediated activity.

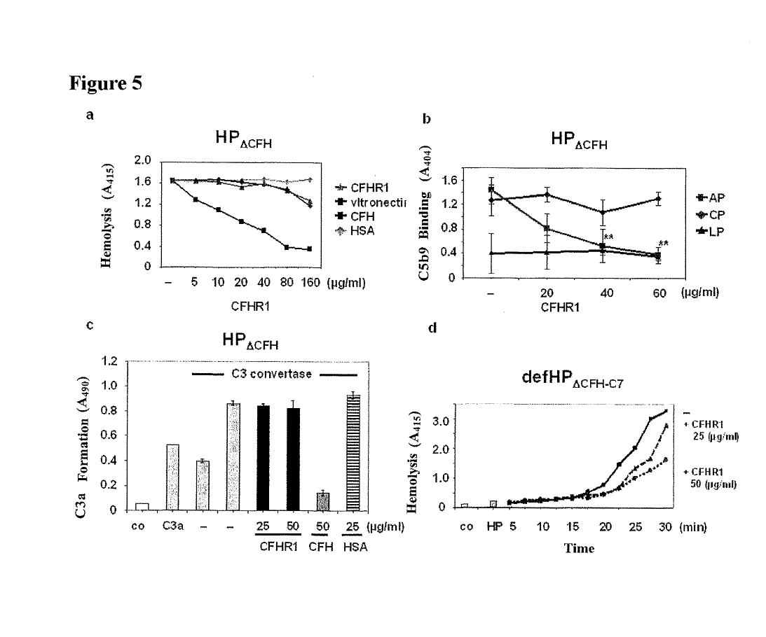

FIG. 4 illustrates the property of CFHR1 of regulating activation of the alternative complement pathway. CFHR1 and the known complement inhibitor Vitronectin exhibited similar inhibitory effects. In contrast to CFH. An action on the classical pathway or the lectin pathway was not apparent, however. CFHR1 here does not act on C3 convertase, see FIG. 5. However, CFHR1 was found to have a dose-dependent inhibitory effect on complement-mediated lysis. That is to say, CFHR1 affects the formation and activity of the MAC.

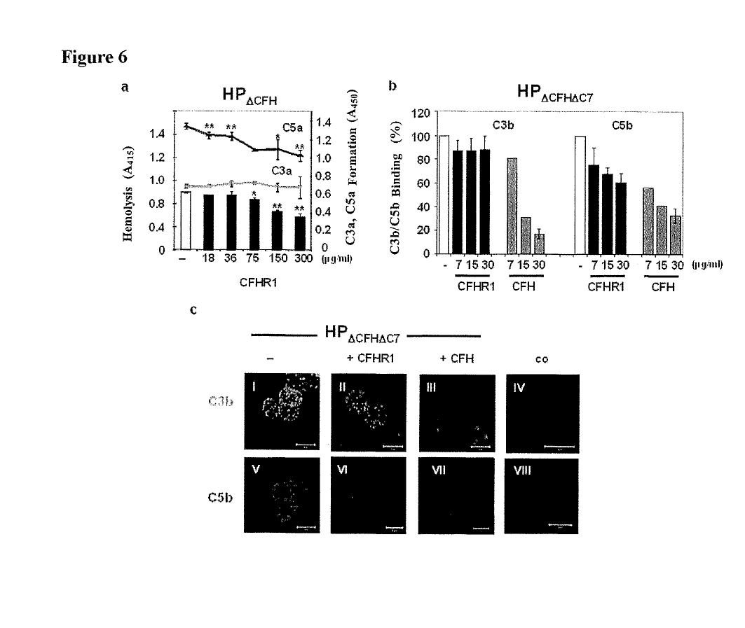

As illustrated in FIG. 6, CFHR1 regulates C5 convertase of the alternative pathway. The molecule has an inhibitory effect on the formation of C5b but not on C3b, and can inhibit C5a generation, also in a dose-dependent manner, see FIG. 6a.

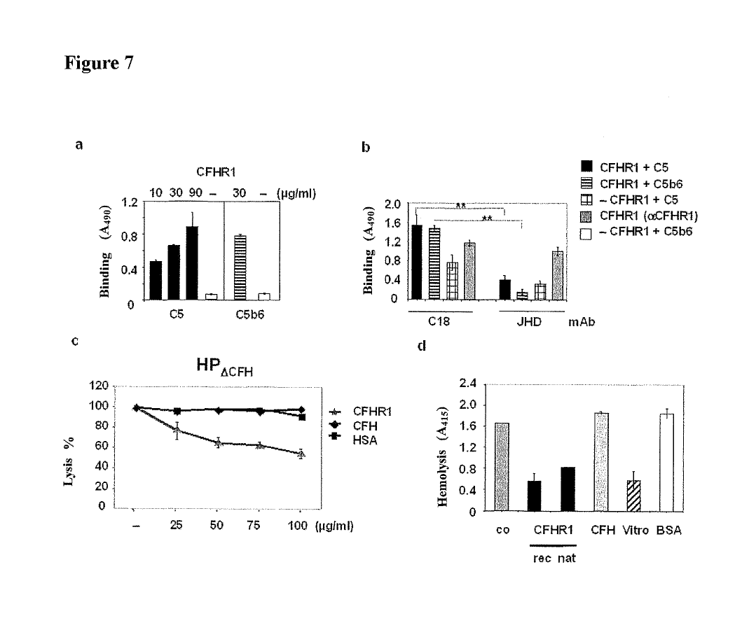

FIG. 7 illustrates that CFHR1 binds to C5 and C5b6, with the N-terminal region of the molecule. CFHR1 here prevents formation of the MAC, see FIG. 7c. In order to confirm that this effect cannot be attributed to recombinant artifacts, native purified CFHR1 protein was used to confirm the effects, see FIG. 7d.

1. Use of a CFHR molecule, in particular a CFHR protein, or a functional fragment or functional derivative thereof, the latter inhibiting the enzyme C5 convertase, for the treatment or prophylaxis of autoimmune diseases and inflammatory reactions.

2. Use according to claim 1, wherein the inflammatory reaction is an inflammatory reaction in the course of an autoimmune disease.

3. Use according to claim 1, wherein the inflammatory reaction is a reaction induced in the course of a sepsis, rheumatoid arthritis, Alzheimer's disease, atheriosclerosis, lupus erythematosus or antiphospholipid syndrome.

4. Use according to claim 1, wherein the CFHR protein or a functional fragment or a functional derivative thereof prevents formation of the terminal membrane attack complex.

5. Use according to claim 1, wherein the alternative complement activation pathway is influenced, more specifically inhibited.

6. Use of a CFHR protein or of a functional derivative or of a functional fragment thereof for inactivating the complement activation, in particular during transplantation or dialysis.

7. Use of a CFHR protein or of a functional fragment or of a functional derivative thereof for coating implants and surfaces which may come into contact with body fluids.

8. Use according to claim 1, wherein the CFHR protein is the CFHR1 protein.

9. Coating for devices coming into contact with body fluids, in particular plasma or blood, characterized in that the surface of said coating comprises CFHR protein, a functional fragment or functional derivative thereof.

10. Coating according to claim 9, characterized in that it is for an implantable device, said implantable device being intended for use in the body of an individual.

11. Device coming into contact with body fluids and in particular blood, more specifically implantable device, characterized in that CFHR protein, a functional derivative or functional fragment thereof has been applied to the surface.

12. Pharmaceutical composition comprising functional CFHR protein in combination with functional factor H and optionally with further pharmaceutically acceptable diluents, carriers and excipients.

13. Pharmaceutical composition according to claim 12, comprising functional CFHR1 protein.

14. Monoclonal antibody which specifically detects a CFHR protein immunohistologically and immunobiochemically and which can specifically bind to human CFHR, more specifically CFHR1 protein, wherein said monoclonal antibody has the same properties as the monoclonal antibody of the JHD-7.10.1 hybridoma cell line, deposited in accordance with the Budapest Treaty with the DSMZ, Brunswick, Germany, DSM ACC 2978.

15. Monoclonal antibody according to claim 14, which can specifically bind to human CFHR protein, wherein said monoclonal antibody reacts with the same epitope of human CFHR protein as the monoclonal antibody that can be obtained from the JHD-7.10.1 hybridoma cell line, deposited in accordance with the Budapest Treaty with the DSMZ, Brunswick, DSM ACC 2978.

16. Monoclonal antibody according to claim 14, characterized in that it does not react with complement factor H.

17. Hybridoma cell line expressing a monoclonal antibody according to claim 14.

18. Method of determining CFHR molecules, more specifically CFHR1 protein, in body fluids, comprising the step of incubating a sample to be tested with a monoclonal antibody according to claim 14, and determining CFHR molecules bound to said antibody.

19. Method according to claim 18 for determining the CFHR1 content in a body fluid of an individual for diagnosing hemolytic uremic syndrome, age-related macular degeneration, or of membranoproliferative glomerulonephritis and other inflammation-based renal disorders.

20. Method according to claim 20 for diagnosing hemolytic uremic syndrome, further comprising the step of determining autoantibodies in a body fluid of the individual.

21. Method according to claim 18, wherein the body fluid is blood, plasma or serum.
